(19) United States
(12) Patent Application Publication
(10) Pub. No.: US 2025/0388490 A1
(43) Pub. Date: Dec. 25, 2025

(54) SHOWER HEAD WITH UVC LEDS TO DISINFECT THE WATER AND THE SHOWER HEAD

(71) Applicant: Canopus Water Technologies Inc., Windham, NH (US)

(72) Inventors: Souheil Benzerrouk, Windham, NH (US); Ronald Walter Collins, Londonderry, NH (US)

(21) Appl. No.: 18/612,442

(22) Filed: Mar. 21, 2024

Related U.S. Application Data
(60) Provisional application No. 63/453,490, filed on Mar. 21, 2023.

(51) Int. Cl. C02F 1/32; B05B 1/18

(52) U.S. Cl. CPC C02F 1/325; B05B 1/18; C02F 2201/009; C02F 2201/3227; C02F 2201/3228; C02F 2303/04; C02F 2307/06

(57) ABSTRACT

A shower head with integrated water disinfection and fixture sanitation. The shower head includes two sections, a first section to treat incoming water and a second section to sanitize the fixture, more specifically the areas within the shower head with the potential for bacterial growth including the shower head aerator and spray/jet ports. The sections are separated by UV reflective material such as PTFE or employ a PTFE coating or liner on the two sections to increase UV reflections and diffusion through the volume of both sections, especially the second section. UV LEDs are positioned in specific locations to both provide maximum UV intensity in the main water flow path and allow enough reflections to fill the reactor with UV. The shower head is scalable in size and in the number of LEDs employed. The LEDs may be powered by a water flow driven dynamo and/or a battery.

### CROSS-REFERENCE TO RELATED APPLICATIONS

This application claims priority from US Provisional Patent application number 63/453, 490 filed Mar. 21, 2023 titled A Shower Head with UVC LEDs to Disinfect the Water and the Shower Head, which application is fully incorporated herein by reference.

### TECHNICAL FIELD

The present invention relates generally to non-chemical water disinfection and more particularly, to utilizing ultraviolet light in the germicidal range to inactivate bacteria and micro-organisms in shower water and prevent bacterial growth in the shower head.

### BACKGROUND INFORMATION

Water disinfection is an integral part of any water treatment system including drinking water, industrial process water, and wastewater. There has been a recent increase in the occurrence of legionnaire disease in hot tubs and showers in large facilities such as hospitals, gyms, offices, and hotels. Legionnaires disease is caused by the presence of the Legionella, a bacterium in the water, in fixtures or in a stagnant water. Shower heads in particular present a challenge due to the shape, size and complexity due to aerators and other design elements that can prevent the shower head or the hose to completely drain, thereby promoting the growth of the Legionella bacteria.

Recently, UV LEDs in the germicidal range have been introduced with power levels that can provide sufficient radiation to inactivate Legionella and other bacteria that can be present in water or grow in a fixture. LEDs have the benefits of longer life (more than five times that of a traditional lamp) and the ability to be turned ON/OFF instantaneously allowing them to be used in a shower.

### SUMMARY OF THE INVENTION

A shower head that includes one or more UVC LEDs. The said shower head is constructed with two sections separated by divider plate. A first set of LEDs are positioned in specific locations in the first section to provide disinfection in the main flow path of shower water. A second set of LEDs are positioned in the second section to disinfect the showerhead aerator and the water spray ports. Separation is accomplished using a standard material or as in one of the embodiments, a PTFE divider to ensure UVC light from the UVC LEDs is diffused in both sections. The shower head is scalable in size and in the number of LEDs employed.

Several embodiments are envisioned for various applications such as: a battery back-up to power the LEDs, and a dynamo to power the LEDs and charge the battery when used. Also, a communications system that allows the owner of the disinfection system to monitor the system remotely and receive status updates or notifications. The two main embodiments include a first embodiment where a main disinfection chamber and an outlet section which may include the aerator separated by a divider plate as shown in FIG. 1 and FIG. 2. This divider plate can be made with PTFE or other UV reflective material to further reflect/diffuse the UVC in each chamber. Second main embodiment shown in FIG. 3 where the two section are in series such that a first water disinfection chamber ensures the incoming water is treated prior to exiting through the outlet chamber which may include an aerator which section also includes LEDs to keep this portion of the shower head free of bacteria when water is not flowing through the shower head.

In one embodiment, the one or more LEDs may be of the same output power while in another embodiment, the one or more LEDs in the main flow section may operate at a higher power either continuously or on-demand to achieve the desired dose for the flow rate while the one or more LEDs in the second chamber may operate continuously at a lower power to prevent bacterial growth.

### BRIEF DESCRIPTION OF THE DRAWINGS

These and other features and advantages of the present invention will be better understood by reading the following detailed description, taken together with the drawings wherein:

FIG. 1 is a partial cutaway view of the showerhead according to the present invention with two disinfection chambers separated by a divider, each chamber including one or more UVC LEDs to treat the influent water for the main chamber and to sanitize the aerator and outlet ports and provide additional water disinfection for the second chamber;

FIG. 2 is a cross-section of the preferred embodiment showing the construction with the two showerhead chambers superposed above one another, separated by a divider plate, and illustrating a dynamo to provide power to the LEDs and charge the battery; and

FIG. 3 is a cross-section of a second embodiment where the two chambers are in series, with each chamber including one or more UVC LEDS.

### DETAILED DESCRIPTION OF THE PREFERRED EMBODIMENTS

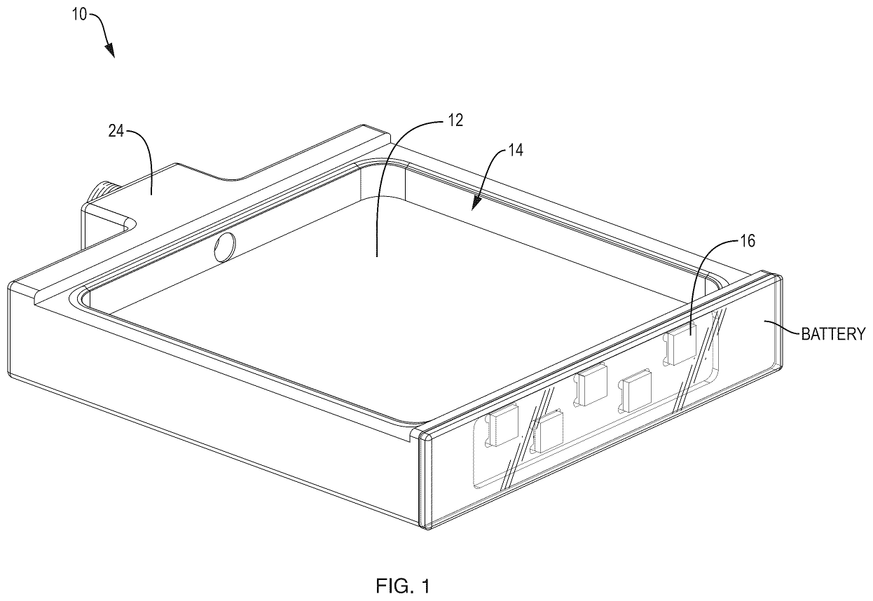

A showerhead 10 that both disinfects influent water and sanitize itself including the aerator if included, FIG. 1, includes two chambers (only the first chamber 14 shown in this view) separated by a divider 12, wherein each chamber contains at least one or more UVC LEDS 16 (LEDs 16a in the first disinfection chamber 14 and LEDs 16b in the second disinfection chamber 22) to provide the necessary and appropriate amount of UV radiation to the showerhead chambers. UV (Ultra-violet) light represents wavelengths that fall between visible light and x-ray on the electromagnetic spectrum. The UV range can be further divided into UV-A, UV-B, UV-C, and Vacuum-UV. The UV-C portion of the UV spectrum represents wavelengths from 200 nm-280 nm, the preferred wavelength used in LED disinfection products in accordance with the teachings of the present invention.

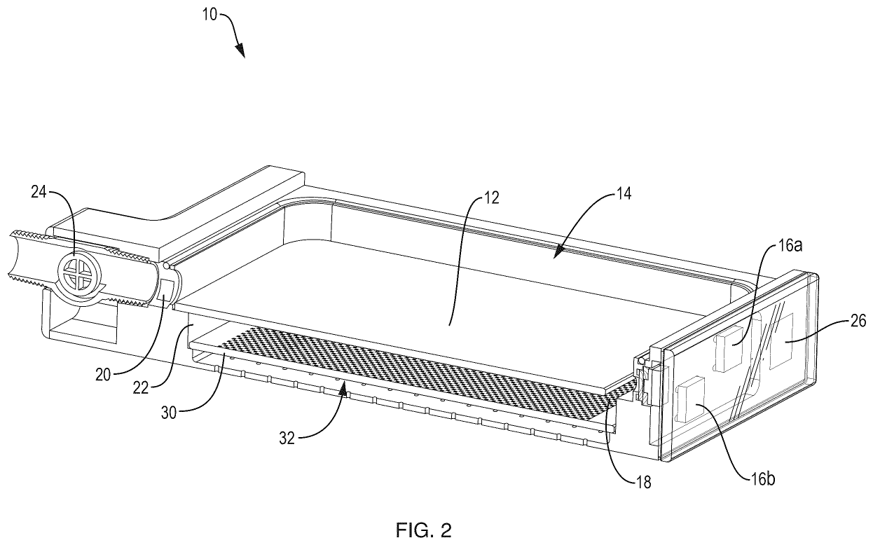

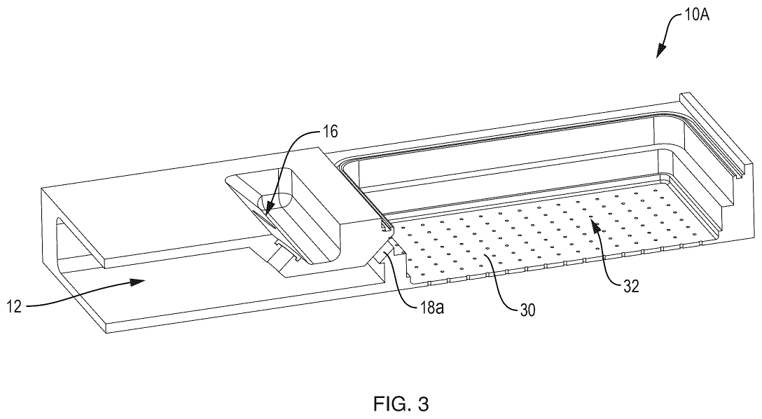

The showerhead 10 is, in one embodiment, constructed of two chambers superposed above one another as shown in FIGS. 1 and 2 and separated by a divider 12 which is preferably made of or covered by a UV reflective or diffusing material such as PTFE (polytetrafluoroethylene). In another embodiment, the interior of the shower head first disinfection chamber 14 may be made from or coated with PTFE. Alternatively, in another embodiment, the showerhead 10 may be constructed wherein the two chambers are in series as shown in FIG. 3. The purpose of the high intensity UV dosage and reflectivity of the shower head chamber interior and/or divider 12 is to help insure that the water flowing through the shower head is exposed to a specific UVC dose sufficient to kill or inactivate common bacteria that maybe present in the water and be found residually in the showerhead chambers and components such as the aerator and jets 30 disposed in the aerator section 32 such as Escherichia Coli (E Coli), Salmonella, Cryptosporidium, or Legionella Pneumophila. Legionella for example is known to grow in showerheads and other fixtures (such as fir example in the aerator 30 and/or aerator section 32. Typically, 16 mJ can reduce these pathogens by more than a 4-log factor or 99.99%.

The desired dose per individual showerhead chamber to achieve optimal desired bacteria kill and disinfection is computed as: d=It measured in mJ/cm2 with I is the UV power density (or intensity) in mJ/cm2 and t is the time. For the main chamber where influent water is treated the requirement is to provide at least a dose of 16 mJ/cm2 to ensure a disinfection (killing or inactivation) of more than 4-log of the most known bacteria. The second chamber and its associated UVC LEDs is designed and configured to ensure the prevention of bacterial growth in the showerhead and specifically in the aerator portion of the shower head.

Extensive experimental studies were conducted to estimate the amount of UVC light intensity/dosage required to achieve a specific reduction (killing and/or inactivation) rate of specific bacteria and viruses. For instance, for Escherichia coli (E-coli) and Legionella, a dose of 10 milli-joules is sufficient to achieve 99.9% reduction.

To ensure that the desired disinfection requirements are met, the first and second chambers are separated by a small gap 18, FIG. 2, that limits the water flow rate and increases the pressure within the first or main chamber 14 hence a better and longer UV transmission/exposure in the water. For the first embodiment this is achieved by ensuring the dividing plate provides a small gap 18 where water flows consistent with the desired flow rate. For the second embodiment shown in FIG. 3, it is a narrow water channel 18a between the first or main disinfection chamber 14 and the second disinfection chamber 22.

Due to the instant ON/OFF capability of the LEDs, the main chamber 14 can operate in an on-demand mode with the addition of a flow sensor or switch 20. Such an approach will further extend the life of the LEDs.

The aerator is sanitized through the use of lower intensity LEDs that are operated continuously or turned on at a predetermined schedule to ensure there is no existing or ongoing bacterial growth in the zone of the second chamber 22.

Controlled operation results in higher efficiency and improved safety, therefore, the system can further include a combination of a dynamo 24 and a battery 26 to isolate the shower head from wall power.

Accordingly, the present invention provides a novel showerhead with built in UVC light disinfection of both a main water chamber as well as a showerhead aerator or jet section to kill any residual bacteria that might be found in the aerator portion of a shower head. The showerhead is self-contained in that the UVC LEDs are powered by a battery and/or a water stream powered dynamo, thus no connection to household current is required.

Modifications and substitutions by one of ordinary skill in the art are considered to be within the scope of the present invention which is not to be limited except by the allowed claims and their legal equivalents.

The invention claimed is:

1. A shower head with UVC disinfection, said shower head comprising:
two UVC disinfection sections, a first UVC disinfection section configured for the disinfection of water entering and leaving said first UVC disinfection section, and a second UVC disinfection section, said second UVC disinfection section configured for the disinfection of the shower head, to prevent the growth of bacteria in shower head and shower head aerator and spray/jet ports.

2. The shower head of claim 1 wherein each of the first and second UVC disinfection sections include one or more UVC LEDS that emit sufficient UVC light intensity in the germicidal UV range so as to disinfect a flow of water flowing through the shower head and prevent growth of bacteria in said shower head and in a shower head aerator and spray/jet ports.

3. The shower head of claim 1 wherein said first and second UVC disinfection sections are separated with a UV reflecting material of an appropriate thickness of a minimum of 1 mm, to ensure more than 90% of the UVC light is reflected, and wherein said UV reflective material is configured to further diffuse the UVC light to provide a better coverage in the shower head aerator and spray/jet ports.

4. The shower head of claim 1 further comprising a small gap between the first and second disinfection sections to ensure an appropriate water flow profile and better UVC light distribution throughout the shower head.

5. The shower head of claim 2 wherein the one or more UVC LEDs are positioned to provide maximum UVC light intensity in a restricted flow area and to cause the UVC LED light to be reflected in a manner that fills the first and second disinfection sections with UV light.

6. The shower head of claim 2 wherein the UVC LEDs are powered from at least one of a shower head integrated dynamo or battery.

7. The shower head of claim 2, wherein the one or more UVC LEDS are of the same output power.

8. The shower head of claim 2 wherein the one or more LEDs in the first disinfection section operate at a higher power either continuously or on-demand to achieve a desired UVC light dose for a water flow rate, while the one or more UVC LEDs in the second disinfection chamber operate continuously at a lower power to prevent bacterial growth in the shower head aerator and spray/jet ports.

9. The shower head of claim 8, wherein the UVC light intensity per individual showerhead chamber is computed according to the formula as: d=It measured in mJ/cm2 with I is the UV power density (or intensity) in mJ/cm2 and t is the time, and wherein in the first disinfection chamber where influent water is treated, the UVC light dosage is at least 16 mJ/cm2 to ensure a disinfection (killing or inactivation) of more than 4-log of the most known bacteria.

10. A shower head with UVC disinfection, said shower head comprising:
two UVC disinfection sections, a first UVC disinfection section configured for the disinfection of water entering and exiting said first UVC disinfection section, and a second UVC disinfection section configured for the disinfection of the shower head, to prevent the growth of bacteria in the shower head and shower head aerator and spray/jet ports;
wherein each of the first and second UVC disinfection sections include one or more UVC LEDs that emit sufficient UVC light intensity in the germicidal UV range so as to disinfect a flow of water flowing through the shower head and prevent growth of bacteria in said shower head and in a shower head aerator and spray/jet ports, and wherein the one or more UVC LEDs in each of the first and second UVC disinfection sections are positioned to provide maximum UVC light intensity in a restricted water flow area and to cause the UVC LED light is reflected in a manner that fills the first and second disinfection sections with UVC light, and wherein the UVC light intensity per individual showerhead chamber is computed according to the formula: d=It measured in mJ/cm2 wherein I is the UV power density (or intensity) in mJ/cm2 and t is the exposure time, and wherein in the first disinfection chamber where influent water is treated, the UVC light intensity dosage is at least 16 mJ/cm2 to ensure a disinfection (killing or inactivation) of more than 4-log of the most known bacteria;
wherein each of the UVC LEDs are powered from at least one of a shower head integrated dynamo or a battery; and
wherein said first and second UVC disinfection sections are separated with a UVC reflecting material of an appropriate thickness of a minimum of 1 mm, to reflect the UVC light from the UVC LEDS.

11. The showerhead of claim 10, wherein said UV reflecting material is configured to further diffuse the UVC light to provide more UVC light to the shower head aerator and spray/jet ports.

12. The showerhead of claim 10, further comprising a small gap between the first and second disinfection sections to ensure an appropriate water flow profile and better UVC light distribution throughout the shower head.

13. The showerhead of claim 10, wherein the one or more UVC LEDs are each of the same output power.

14. The showerhead of claim 10, wherein the one or more UVC LEDs in the first disinfection section operate at a higher power level either continuously or on-demand to achieve a desired UVC light dose for a given water flow rate, while the one or more UVC LEDs in the second disinfection section operate continuously at a lower power level to prevent bacterial growth in the shower head aerator and spray/jet ports.
